(19) United States
(12) Patent Application Publication
(10) Pub. No.: US 2026/0120594 A1
(43) Pub. Date: Apr. 30, 2026

(54) Functional Phantom System

(71) Applicant: Biopixs Ltd, Cork (IE)

(72) Inventors: Sanathana Konugolu Venkata Sekar, Cork (IE); Claudia Nunzia Guadagno, Milano (IT); Stefan Andersson-Engels, Lancaster Gate, CO (US)

(21) Appl. No.: 19/481,858

(22) PCT Filed: May 3, 2024
(86) PCT No.: PCT/EP2024/062259

§ 371 Date: Jan. 13, 2026

(30) Foreign Application Priority Data
May 5, 2023 (GB) 2306700.2

(51) Int. Cl. G09B 23/28; A61B 5/00

(52) U.S. Cl. CPC G09B 23/286; A61B 5/0075; A61B 2560/0223

(57) ABSTRACT

Disclosed is a tissue phantom system, comprising: a tissue phantom material having known optical properties and an input surface for receiving incident source light from the optical measurement instrument; and one or more light guides having a first and second end, wherein the first end of the or each light guide is embedded within the phantom material at a respective location beneath the input surface to transmit light to and/or from the respective location for simulating an optical property variation within the tissue phantom material. Also disclosed is a method of calibrating or testing an optical measurement instrument configured to measure a target's response to illumination, by: illuminating the input surface of the tissue phantom material with source light, simulating an optical property variation within the tissue phantom material according to a known response pattern by transmitting light to and/or from the respective location beneath the input surface via the one or more light guides; and detecting scattered light emitted from the tissue phantom material.

### TECHNICAL FIELD

This invention relates generally to tissue mimicking phantoms for use in testing and calibrating optical instruments and the standardisation of bio-optical techniques, and particularly to functional phantoms designed to mimic the dynamic optical properties of tissue.

### BACKGROUND TO THE INVENTION

The development of new optical medical diagnostic tools introduces a compelling need for calibration and standardization, in particular for simple and reliable ways to assess the quality of optical measurement instruments. Various different quality assessment protocols have been developed, the most important and widely adopted of which being the “BIP Protocol”, the “MEDPHOT Protocol”, the “nEUROPt Protocol” and the functional near infrared spectroscopy (fNIRS) protocol IEC 80601-2-71, as are known in the art. In all cases, protocols involve the use of tissue mimicking phantoms (referred to herein as tissue phantoms) with known optical properties including scattering and absorption to assess the characteristics of the optical measurement instruments.

Tissue phantoms have been routinely used since the 1980s, when interest in optical or near-infrared (NIR) spectroscopy and imaging of tissue began to surge. Since then, the introduction of spatially resolved, time-resolved and frequency-domain light sources, and continued research and development of new biomedical applications of light has cemented the importance of tissue phantoms and lead to the development of many different types of tissue phantoms, including solid, water-based and gel-based phantoms with different absorbers, scatterers and additives. Well calibrated tissue phantoms are now essential for routine system comparison, evaluation and quality control, driving the need for improvement and standardization of tissue phantoms.

Traditional tissue phantoms are intentionally “static” in nature, consisting of a scattering medium with fixed and stable optical properties. The development and clinical/commercial success of modern functional NIR (fNIR) spectroscopy tools for monitoring and measuring dynamic phenomena, including but not limited to hemodynamics, has generated a need of “dynamic” functional tissue phantoms whose optical properties can be varied or configured in a way that mimics the time-varying optical properties of the target tissue.

Various different solutions have been proposed so far. Arguably, the most practical and commercially viable solutions are those with optical properties that can be electronically modulated. It is known to embed electrical devices within a tissue phantom. For example, US 2005/145786 A1 discloses a tissue phantom with an embedded light source for use in calibrating low-level light in vivo imaging systems. Building on this, various examples of tissue phantoms with embedded electrically controllable absorber/scatterer elements have been reported. EP 1726256 A1 discloses a dynamic solid tissue phantom for biomedical imaging with one or more embedded segmented liquid crystal devices (LCDs). The segmented LCD forms a graphical layout of anatomical structures such as blood vessels, the opacity of which can be electronically modulated to vary the absorption in the segments and mimic dynamical properties of the anatomical structure. Multiple electronic device layers can be embedded in tissue layers to mimic the 3D properties of an organic object such as an arm or the like. US 2007/0004026A1 discloses a dynamic solid tissue phantom with an embedded LCD that is electronically modulated with a wavelength-dependent time-varying drive voltage to attenuate incident light propagating within the surrounding scattering medium according to a known hemodynamic response. Similar approaches of embedding LCDs with the phantom are also described in, R.L. Barbour et al. “Validation of near infrared spectroscopic (NIRS) imaging using programmable phantoms” Proc. Of SPIE Vol. 6870 687002(2008); and R.L. Barbour et al. “A programmable laboratory testbed in support of evaluation of functional brain activation and connectivity”, IEEE transactions on neural systems and rehabilitation engineering, Vol. 20 No. 2 Mar. 2012.

The use of electrochromic devices such as LCDs is an attractive solution for mimicking hemodynamic (and other dynamic) behaviour due to their fast switching time (e.g. <10 ms), low power consumption, long term stability, and lack of a memory effect. Embedded segmented LCDs also provide the opportunity of mimicking anatomical structures such as vessels plexus (e.g. see EP 1726256 A1). However, electrochromic materials, in particular LCDs, are designed to work optimally with normal incident light and exhibit strong angle dependence of its attenuation properties. Due to the highly diffuse nature of light propagation with the phantom material, light is scattered in all directions such that light impinging on the electrochromic device has a no well-defined angle or distribution. This impacts the performance and predictability of the phantom, as certain angles modulate the light while others do not, and some angles modulate in the opposite direction. An LCD will have a different response dependent on how deep it is embedded in the phantom due to change from ballistic to diffusive scattering within the phantom. The modulation behaviour further changes as a function of the optical properties (absorption and scattering) of the surrounding phantom material, and any other instrument factors that affect the light distribution in the phantom, such as spot size. All of this makes it hard to predict the performance and effective attenuation created by an embedded LCD in use, and with different instruments, which is adverse to the goal of improving standardisation.

Alternative solutions for providing versatile and well-calibrated functional phantoms are therefore needed. In particular, there is a need for improved functional phantoms that can simulate/mimic spatial and/or temporal variations in the optical properties of tissue and that are suitable for calibrating, testing and standardisation of a broad range of spectroscopic tools. Aspects and embodiments of the present invention have been devised with the foregoing in mind.

### SUMMARY OF THE INVENTION

According to a first aspect of the invention, there is provided a tissue phantom system for calibrating or testing an optical measurement instrument. The system may be referred to as a functional phantom system. The optical measurement system may be configured to measure a target's response to illumination, e.g. by illuminating the target with source light and detecting scattered light emitted from the target with spatial and/or temporal resolution. The tissue phantom system comprises a tissue phantom material with an input surface for receiving incident source light from the optical measurement instrument. The phantom material may comprise one or more known static/background optical properties, such as absorption and/or scattering. The tissue phantom system further comprises one or more light guides that are at least partially embedded within the phantom material. The or each light guide has a first end and a second end and is configured to transmit light therebetween. The first end of the or each light guide is embedded within the phantom material at a respective location beneath the input surface. The one or more light guides are configured to transmit light to and/or from the respective location(s) within the phantom material. The first ends of the one or more light guides may couple light out of the phantom material (e.g. at least a portion of incident source light propagating within the phantom material) and/or couple light into the phantom material (e.g. light transmitted from an optical arrangement, see below). In this way, by controlling the light propagating within the tissue phantom material, a desired optical feature such as an optical property/optical property variation (which is generally localised to the respective location(s) of the first end(s)) within the phantom material can be simulated.

Using embedded light guides to simulate optical property variations within the phantom material provides a number of technical advantages over, and removes all the variabilities associated with, prior art approaches with embedded liquid crystal devices (LCDs). In particular, light is collected and/or emitted from the first end of the light guide in a predictable way, as determined by the numerical aperture of the light guide. Further, the light collected at the first end is emitted from the second end of the light guide in a predictable way. As such, the response of the light guide is independent of depth within the phantom material, and all light collected by the first end of the light guide can be treated, manipulated or modulated in the same way, whereas in prior art approaches, the angle of the light hitting the LCD effects the attenuation etc. Light collected from the location of the first end can be transmitted to any location inside or outside of the phantom material with negligible attenuation, preferably to an optical arrangement for modulating the light. The embedded light guide approach requires only the first end or tip to be present at the location where you want to produce the optical property variation. The first ends of the light guides can be located in any desired position, pattern or spatial distribution to create the desired spatial response pattern. By contrast, prior art approaches require the embedded LCD or other modulator to be located at the point of modulation. Such modulators take up more space which prevents complete mimicking of tissue features (e.g. an LCD usually includes two polarizers, a liquid crystal, and electrical connections). It is also difficult to produce any specific shape of the optical property variation with an LCD, such as blood vessels and tissue structure. By contrast, any shape can be produced using embedded light guides, e.g. by distributing and/ot bundling the light guides. Thus, using embedded light guides to transmit light to and/or from the respective locations in the phantom material can provide greater design freedom on the type and shape of tissue optical features that can be simulated.

The second end of the or each light guide may be coupled/couplable or connected/connectable to an optical arrangement. The system may comprise the optical arrangement. The optical arrangement is configured to receive input light from and/or send output light to the phantom material via the one or more light guides to simulate a desired optical property variation within the phantom material.

The first end of the or each light guide may be configured to collect input light propagating within the phantom material for transmitting to the optical arrangement, and/or emit output light transmitted from the optical arrangement into the phantom material. The optical arrangement may be configured to produce output light and couple the output light into the second end of at least one of the one or more light guides for transmitting to the phantom material.

The simulated optical property variation may be static or temporal/time-varying. Further, because the simulated optical property variation is generally localised to the respective location(s) of the first end(s), the light guide(s) provide a spatial variation in the optical property.

In one preferred embodiment, the optical arrangement and the second end of the or each light guide are located outside of the phantom material. Using the embedded light guide(s) to transmit light to and from an external optical arrangement opens up a myriad of opportunities for using more sophisticated optical arrangements coupled to the second end of the light guide(s) to simulate more complex optical features within tissue which are not possible with prior art approaches of embedding modulating elements within the phantom. In particular, the optical arrangement is not restricted in size or fixed in its configuration. Rather, the optical arrangement can include any optical elements, including active and/or passive optical modulating elements, mirrors, lenses, beam splitters, filters, polarisers, etc. as are standard in table-top optical instruments, and can be reconfigured without damaging or destroying the phantom material and light guides. This makes the phantom system versatile, configurable and suitable for use with a broad range of optical measurement techniques including but not limited to functional near infrared (NIR) spectroscopic and other biophotonic techniques such as diffuse optical spectroscopy, diffuse correlation spectroscopy, fluorescence, hyperspectral and diffuse-reflectance imaging, Raman spectroscopy, optical coherence tomography and photodynamic therapy, based on CW, time domain or frequency domain technologies.

In another preferred embodiment, the optical arrangement and the second end of the or each light guide are also embedded (or at least partially embedded) within the phantom material. This may be suitable for applications where a more compact system is required, or for use with time of flight (ToF) technologies where the length of the light guides can be minimised. For example, where the optical arrangement is also embedded, the length of the or each light guide is preferably less than 5 cm.

The optical arrangement can be configured to simulate at least one of the following optical properties: absorption, scattering, emission, fluorescence, bioluminescence, Raman scattering, and dynamic lighting scattering. The simulated optical property may be static or time-varying.

The optical arrangement can be configured to modulate the output light according to a predefined temporal response pattern. In this way, the optical arrangement can simulate a temporal variation in an optical property of the phantom material. The known/predefined temporal response pattern may be or comprise a hemodynamic response pattern such as a photoplethysmographic (PPG) signal.

The optical arrangement may comprise one or more light sources. The light source(s) can be used for producing the output light that can be selectively coupled into the second end of the or each light guide to simulate the desired optical property variation.

Alternatively or additionally, the light source(s) can be used to produce locator light that can be selectively coupled into the second end of the or each light guide and emitted at the respective first end(s) to indicate the location of the respective first end(s). In this way, when locator light is coupled to the second ends of the light guides, the first ends serve as optical beacons indicating their locations for alignment of the optical measurement instrument. The locator light may be visible light. The light sources may comprise a laser diode or light emitting diode, or other suitable electrically controllable light source. The light source(s) are configured to produce the output light and/or the locator light in response to an input signal.

In a preferred embodiment, the optical arrangement is configured to: receive input light from the second end of a first one of the one or more light guides; modulate or manipulate or otherwise use the input light to produce the output light; and couple the output light into the second end of the first one or a second one of the one or more light guides for transmitting to the phantom material.

The optical arrangement can comprise a reflective element for directing the output light back to the second end of the first one or the second one of the one or more light guides.

The optical arrangement preferably comprises one or more optical modulating elements having a fixed or variable optical property. The one or more optical modulating elements are configured to produce the output light by the interaction of incident input light with the respective optical modulating element.

The one or more optical modulating elements may comprise an active optical modulating element having an electrically controllable optical property. The active optical modulating element may be configured to selectively attenuate incident input light according to an input drive signal to thereby provide/produce the output light. The input drive signal may be or comprises a predefined static or temporal response pattern.

The active optical modulating element may comprise an electrochromic material configured to vary the transmission of incident input light according to an input drive signal. In this case, the transmitted light is the output light.

The active optical modulating element may be or comprise a liquid crystal device (LCD).

The active optical modulating element may be or comprise an electro-mechanical modulator that is configured to vary the reflectance of incident input light according to an input drive signal. In this case, the reflected light is the output light. Preferably, the electro-mechanical modulator is or comprises a deformable mirror device.

The active optical modulating element may be or comprise a mechanical shutter device that is configured to vary the transmission of incident input light according to an input drive signal. In this case, the transmitted light is the output light. Preferably, the mechanical shutter device is configured to selectively transmit or attenuate the incident input light.

Using a deformable mirror device or a shutter device may in principle allows up 100% of light to be modulated and sent back to phantom (as opposed to LCDs which lose light through polarizers). Electro-mechanical modulator and shutter devices are particularly well suited for embodiments with an external optical arrangement.

Preferably, the optical arrangement comprises one or more collimators for coupling input light out of and/or output light into the second end of the or each respective light guide. Alternatively, the second end of the or each light guide can comprise a (integrated) collimator for coupling input light out of and/or output light into the light guide and producing the collimated input light.

Preferably, the active optical modulating element is arranged to receive the collimated input light at normal incidence. This may increase the modulating performance of the optical modulating element, in particular for LCDs that are designed to work optimally with normal incident light and exhibit strong angle dependence of its attenuation properties.

In one embodiment, the one or more light guides comprises a plurality of light guides and the active optical modulating element comprises a segmented liquid crystal device. The second end of each light guide can be arranged to provide input light to a different segment or group or segments of the liquid crystal device. Each segment or a group of segments of the segmented liquid crystal device may be individually addressable and configured to vary the transmission of incident input light thereon according to a separate input drive signal. The segmented liquid crystal device is configured to selectively attenuate incident input light received from the second end of each light guide in response to a respective input drive signal applied to each respective segment or group of segments. Preferably, the optical arrangement comprises a collimating lens array for coupling input light out of the second end of each respective light guide and the segmented liquid crystal device is arranged to receive collimated input light from each light guide at normal incidence.

Each lens of the lens array may be coupled to, or arranged to receive input light from, the second end of different one of the plurality of light guides in order to produce the collimated input light. The collimating lens array may be provided on or over the segmented liquid crystal device.

The optical arrangement can comprise a reflective element arranged to direct output light transmitted through the segmented liquid crystal device back through the segmented liquid crystal device and the collimating lens array for coupling the output light back into the second ends of the light guides.

In addition to or instead of an active optical element, the one or more optical modulating elements may comprise a passive optical modulating element. The passive optical modulating element may include an optical medium configured to produce scattered and/or emitted output light from the interaction of incident light with the optical medium.

The optical medium may be or comprise: a Raman active material, a fluorescent material, luminescent material, a diffuse correlation spectroscopy solution, or a biological material or fluid. The biological material or fluid may be or comprise blood.

In one implementation, the one or more optical modulating elements comprises an active and a passive optical modulating element, and the optical medium is arranged to receive the output light from the active optical modulating element. In this case, light produced from the optical medium is the output light that is transmitted to the phantom material.

Where the input light comprises a plurality of wavelengths, the optical arrangement may comprise a plurality of optical modulating elements for modulating the input light at each respective wavelength. In this case, the optical arrangement may comprise a plurality of spectral filters, one for each optical modulating element, to select the wavelength of input light incident upon each respective optical modulating element.

The system may comprise a driver configured to apply an input drive signal to the (or each) active optical modulating element to produce the output light according to a predefined static or temporal response pattern.

Where the optical arrangement comprises a plurality of optical modulating elements for modulating the input light at each respective wavelength, the driver can be configured to operate in a first mode, and apply an input drive signal to each active optical modulating that is specific to the selected wavelength.

Alternatively, wherein the input light comprises a plurality of wavelengths, the driver can be configured to operate in a second mode, and apply an input drive signal to the active optical modulating element that is a wavelength-dependent time varying drive signal based on a synchronising signal received from the optical measurement instrument. In this way, the modulation of the incident input light provided by the active optical modulating element is synchronised with a detection timing of the optical measurement instrument. In this mode, spectral filters for selecting a wavelength can be omitted and a single active optical modulating element can be used to modulate input light containing multiple wavelengths.

The system may further comprise a processor configured with a computer program to determine the drive signal to be applied to the active optical modulating element based on one or more input parameters including at least one of the following: at least one wavelength of incident light, at least one optical property of a target, and a known static or temporal response pattern. The processor may be integrated with the driver or part of a computing device in communication with the driver.

In various embodiments, the system comprises a plurality of said light guides. In this case, the first ends of each light guide may be located at different respective positions in the phantom material. Alternatively or additionally, the first ends of each light guide may be located at a range of different depths beneath the input surface of the phantom material. The first ends of the light guides may be located at a depth of between 0.01 to 4 cm. Alternatively or additionally, the first ends of each light guide may be spatially distributed according to a predefined spatial response pattern, such as a vessels plexus pattern. Alternatively or additionally, the plurality of light guides may have a range of different core diameters.

The plurality of light guides may be arranged in one or more groups. The first ends of each light guide in a group may be substantially co-located at a point within the phantom material. Optionally or preferably, the or each group of light guides can be configured as a fan-out bundle, whereby the first ends of the group are part of a common end of the fan-out bundle.

The or each light guide may be or comprises: an optical fiber, an optical fiber bundle, or a liquid light guide. The or each light guides may have a numerical aperture of at least 0.2.

The phantom material may comprise an instrument response function (IRF) measurement zone. The IRF measurement zone may comprise a reflective element provided at or on the input surface of the phantom material for reflecting incident source light. Alternatively, the IRF measurement zone may comprise an embedded light guide having a first end positioned at a first location at the input surface of the phantom material for collecting incident source light and a second end positioned at a second location at the input surface for emitting source light transmitted from the first location.

According to a second aspect of the invention, there is provided a method of calibrating or testing an optical measurement instrument. The method comprises: illuminating an input surface of a tissue phantom material with source light, the phantom material having a first end of one or more light guides embedded therein at a respective location beneath the input surface; simulating an optical property variation within the phantom material by transmitting light to and/or from the respective location via the one or more light guides; and detecting scattered light emitted from the tissue phantom material.

The method may further comprise determining a spatial and/or static or temporal response pattern associated with the simulated optical property variation based on the detected light.

The optical property variation may be simulated according to a known response pattern, such as a spatial and/or static or temporal response pattern. The method may further comprise comparing the determined response pattern to the known response pattern.

The method may comprise measuring an instrument response function (IRF). Measuring the IRF may comprise illuminating a reflective element provided at or on the input surface of the phantom material with source light, and detecting the source light reflected by the reflective element. The reflective element may be embedded in the input surface of the phantom material, or may be a reflective coating/surface applied to the input surface of the phantom material. Alternatively, measuring the IRF may comprise illuminating a first end of an IRF light guide provided at a first location at the input surface of the phantom material with source light, and detecting the source light emitted from a second end of the light guide provided at a second location at the input surface of the phantom material. In this case, measuring the IRF may comprise collecting at least a portion of incident source light at the first end of the IRF light guide, transmitting the collected source light to the second end, and detecting source light emitted from the second end of the IRF light guide.

The method may further comprise determining one or more calibration factors for the optical measurement instrument based on the comparison of the determined response pattern and the known response pattern and/or based on the measured IRF. The method may further comprise calibrating the optical measurement instrument using the calibration factors.

The calibration factors may correct for one or more of: accuracy, linearity, stability, reproducibility of the instrument.

Simulating the optical property variation within the tissue phantom may comprise: producing, at an optical arrangement coupled to the second end of the or each light guide, output light; and transmitting the output light to the phantom material via the via one or more light guides.

Simulating the optical property variation within the tissue phantom may further comprise: receiving, at the optical arrangement, input light from the second end of a first one of the one or more light guides; modulating, manipulating or otherwise using the input light to produce the output light; and coupling the output light into the second end of the first one or a second one of the one or more light guides for transmitting to the phantom material.

Coupling the output light into the second end of the first one or the second one of the one or more light guides may comprise reflecting the produced output light back to the second end of the first one or the second one of the one or more light guides.

Preferably, modulating the input light comprises illuminating one or more optical modulating elements with the input light at normal incidence to produce the output light by the interaction of the incident input light with the respective optical modulating element, the one or more optical modulating elements having a fixed or variable optical property.

The one or more optical modulating elements may comprise an active optical modulator having an electrically controllable optical property. In this case, modulating the input light may comprise: applying a static or time-varying input drive signal to the active optical modulating element to selectively attenuate incident input light according to a known static or temporal response pattern.

The one or more active optical modulating elements comprise a liquid crystal device.

The method may comprise using the phantom system according to any combination of features of the first aspect.

According to a third aspect of the invention, there is provided a use of the phantom system of the first aspect to calibrate or test an optical measurement instrument configured to measure a target's response to illumination. The optical measurement instrument may comprise one or more instruments selected from the group including but not limited to: a near infrared spectroscopy (NIRS) instrument, a pulse oximeter, a fluorescence spectroscopy instrument, a Raman spectroscopy instrument, a diffuse correlation spectroscopy instrument, diffuse optical spectroscopy instrument, an optical coherence tomography instrument, gas in scattering media absorption spectroscopy(GASMAS) instrument, a wearable instrument/device, and an implantable instrument/device. The instruments may be continuous wave, time domain or frequency domain instruments.

Features which are described in the context of separate aspects and embodiments of the invention may be used together and/or be interchangeable. Similarly, where features are, for brevity, described in the context of a single embodiment, these may also be provided separately or in any suitable sub-combination. Features described in connection with the phantom system may have corresponding features definable with respect to the method, and vice versa, and these embodiments are specifically envisaged.

### BRIEF DESCRIPTION OF DRAWINGS

In order that the invention can be well understood, embodiments will now be discussed by way of example only with reference to the accompanying drawings, in which:

FIG. 1 shows a schematic diagram of a functional phantom system according to a first embodiment of the invention;

FIG. 2 shows a schematic diagram of an example implementation of the functional phantom system of FIG. 1;

FIG. 3 shows another example implementation of the functional phantom system of FIG. 1;

FIG. 4(a) shows another example implementation of the functional phantom system of FIG. 1;

FIG. 4(b) shows an example wavelength dependent drive signal for the system of FIG. 4(a);

FIG. 5(a) shows another example implementation of the functional phantom system of FIG. 1 with active and passive optical modulating elements;

FIG. 5(b) shows an example configuration of the system of FIG. 5(a);

FIG. 6 shows another implementation of the system of FIG. 1;

FIG. 7 shows a schematic side view of a functional phantom with a plurality of light guides;

FIG. 8 shows a schematic plan view of a functional phantom with a plurality of light guides arranged in a predefined spatial response pattern;

FIG. 9 shows a schematic plan view of a functional phantom with a plurality of light guides arranged in groups;

FIG. 10 shows a schematic diagram of a functional phantom system according to a second embodiment of the invention;

FIG. 11 shows a schematic diagram of an example implementation of the functional phantom system of FIG. 10;

FIG. 12 shows a method of calibrating an optical measurement instrument according to an embodiment of the invention;

FIG. 13(a) and 13(b) show schematic diagrams of a phantom material including an instrument response zone; and FIG. 14(a) and 14(b) show experimental data comparing performance of an embodiment of the invention compared to a prior art embedded LCD system.

It should be noted that the figures are diagrammatic and may not be drawn to scale. Relative dimensions and proportions of parts of these figures may have been shown exaggerated or reduced in size, for the sake of clarity and convenience in the drawings. The same reference signs are generally used to refer to corresponding or similar features in modified and/or different embodiments.

### DETAILED DESCRIPTION

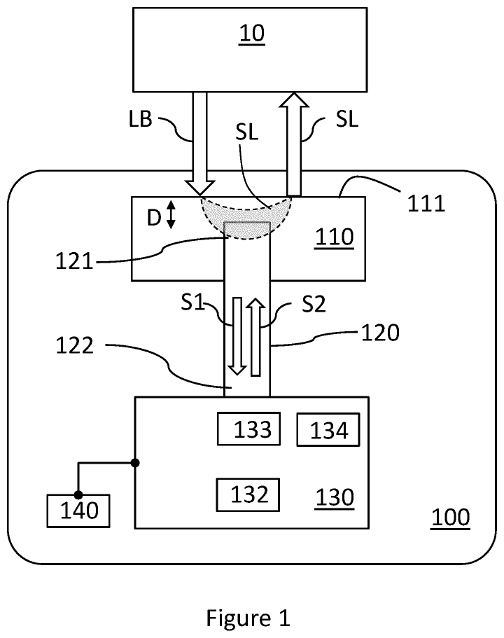

FIG. 1 shows a schematic diagram of a functional phantom system 100 according to a first embodiment of the invention. The phantom system 100 is designed to mimic spatial static and/or temporal variations in optical properties seen in biological tissue for use in testing and/or calibrating biomedical optical measurement instruments 10 that measures a target's response to illumination. Such instruments 10 illuminate the target area of tissue with an input light beam LB in the ultraviolet (UV) to near infrared (NIR), or NIR spectral window (e.g. wavelength ranges 340-2400 nm or 600-2400 nm or 800 to 2400 nm) and detect scattered light SL re-emitted from the target. When light in the NIR spectral window interacts with tissue, it undergoes scattering and absorption dependent on the presence and concentration of certain tissue constituents, known as biomarkers. Biomarkers include but are not limited to: haemoglobin variants such as oxy- and deoxyhaemoglobin, oxygen saturation (arterial, venous and tissue), water, lipids, collagen, melanin, bilirubin, glucose, presence of gas absorption lines (e.g. detected in gas in scattering media absorption spectroscopy(GASMAS)), refractive index, etc. These biomarkers typically vary spatially and/or temporally, particularly those produced by hemodynamic behaviour, producing a distinct spatial and/or temporal response pattern. Provided the instrument is well-calibrated, spatial and/or temporal changes in tissue optical properties (i.e. absorption coefficient μₐ and reduced scattering coefficient μₛ′) can be determined from measurements of scattered light SL from the tissue at certain wavelengths, and various static or dynamic biological/physiological properties of the target can be estimated based on the measured optical properties.

The phantom system 100 is a versatile and configurable tool that can be configured to mimic/simulate spatial and/or temporal variations in a broad range of tissue optical properties (including absorption, scattering, Raman, fluorescence, diffuse correlation etc.) making it suitable for use with a range of optical measurement techniques including functional near infrared (NIR) spectroscopic and other biophotonic techniques (such as diffuse optical spectroscopy, diffuse correlation spectroscopy, fluorescence, hyperspectral and diffuse-reflectance imaging, Raman spectroscopy, optical coherence tomography and photodynamic therapy) based on CW or time of flight (ToF) technologies.

The system 100 includes a phantom material 110 having an input surface 111 for receiving an incident source light beam LB from the optical measurement instrument 10. The phantom material 110 is formed of or comprises a solid scattering medium with known optical properties (e.g. an absorption coefficient μₐ and a reduced scattering coefficient μₛ′) typical of biological tissue. The phantom material 110 includes a mouldable matrix material, such as silicone, polyurethane, epoxy, intralipid (i.e. liquid-based) or hydrogel, and one or more scattering and/or absorbing materials to provide the desired optical properties, as is known in the art. In practice, the choice of phantom material 110 will depend on the desired temporal response, spectral properties, long term stability, reproducibility as well as manufacture ease and cost. In one example, the phantom material 110 is silicone-based with μₐ=0.1 cm⁻¹ and μₛ′=10 cm⁻¹. Incident source light in the NIR spectral window penetrates deep into the phantom material 110 and undergoes absorption and scattering dependent on the optical properties μₐ and μₛ′. Scattered light SL re-emitted from the phantom material 110 is detected by the optical measurement instrument 10 as illustrated in FIG. 1.

The phantom material 110 by itself is static in nature, in that its background optical properties (μₐ, μₛ′) are fixed. To mimic or simulate spatial and/or temporal variations in the optical properties as seen in biological tissue, the system 100 further comprises one or more light guides 120, a first end 121 of which is embedded within the phantom material 110 beneath the input surface 111 and a second end 122 of which is coupled to external optical modulation arrangement 130 located outside of the phantom material 110.

The one or more light guides 120 couple light out of/into the phantom material 110 to/from the optical modulation arrangement 130 to alter or modulate the scattered light SL detected by the optical measurement instrument 10. Specifically, the or each light guide 120 is configured to collect, at its first end 121, input light SI propagating within the phantom material 110 (and within the numerical aperture NA of the light guide) and guide it to the optical modulation arrangement 130 at its second end 122. The optical arrangement 130 is configured to receive the input light S1 from the second end 122 of the respective light guide 120, modulate or manipulate or otherwise use the input light S1 to produce output light S2, and couple the output light signal S2 into the second end 122 of the same or a different one of the light guides 120 for sending back to the phantom material 110 thereby altering or modulating the scattering light SL propagating within the phantom material 110 and detected by the optical measurement instrument 10.

The light guides 120 can be positioned and embedded during the molding process of the phantom material 110. The first end 121 of the or each light guide is located at desired depth, within the penetration depth of the incident NIR light. In one example, the depth is 0.5-1 cm beneath the surface 111. However, it will be appreciated that the specific depth is chosen according to the activation area/tissue feature being mimicked. Further, where there are a plurality of light guides 120, the depths of the first ends 121 can be varied.

The light guides 120, which can be optical fibers, fiber bundles, or a liquid light guides, allow light propagation with negligible attenuation and signal distortion, and can therefore be used to convey light from an initial point to a desired location, in this case from inside the phantom material 110 to outside the phantom material 110 where it can be manipulated by the optical modulation arrangement 130 and then sent back to the phantom 110 to mimic optical property changes.

The use of light guides 120 provides a number of advantages over prior art approaches of embedded electro-active devices. Firstly, it allows external modulation. By routing light out of the phantom material 110 to an external optical modulation arrangement 130 via the light guides 120, light can be manipulated and output light produced in ways that are simply not possible in prior art solutions with embedded LCDs, allowing almost any type of static or dynamic optical phenomenon in tissue to be mimicked. Secondly, it provides greater control and predictability of the modulation properties of the phantom. The light within the phantom material 110, which is randomly scattered in all directions, is collected at the first end 121 of the light guide 120 in a predictable way (depending in the NA of the light guide 120) and emerges from the second end 122 as a regular and predictable beam (angular distribution, profile) which can be precisely controlled and manipulated using conventional optics and bench top methods. In particular, the input light can be transformed into collimated input light beam (see below) to provide precise control over the light propagation direction and angle of incidence on the various components of the optical modulation arrangement. As most optical elements and devices, such as LCDs, filters, polarisers, lenses, etc. are designed to be operated with collimated and normally incident light, this approach allows optimal component performance and predictable light modulation in the phantom to be achieved.

The optical arrangement 130 comprises one or more optical modulating elements 132 having a fixed or variable optical property configured to produce the output light signal S2 by the interaction of the incident input light S1 with the respective optical modulating element 132. The optical arrangement 130 also comprises a collimator 133 for collimating input light S1 coming out of, and/or coupling collimated output light S2 into, the second end 122 of the or each respective light guide 120. The collimator 133 produces a collimated beam of input light S1 for illuminating the one or more optical modulating elements 132 along an illumination optical path. Output light S2 from the one or more optical modulating elements 132 is coupled to the second end 122 of the light guide 120 along a collection optical path, which may be the same or different to the illumination path depending on the arrangement, e.g. whether the same or different light guide is used to send the output light S2 back to the phantom material 110. One or more optical components, such as filters, mirrors, lenses, polarizers, etc. may be positioned in the illumination and/or collection optical path.

Preferably, the one or more optical modulating elements 132 include at least one electrically controllable “active” optical modulating element 132a with a variable optical property configured to selectively attenuate the incident input light beam S1 (by absorption or scattering) according to an applied drive signal Vd received from driver 140. The drive signal Vd is preferably a time-varying signal generated according to a known temporal response pattern seen in tissue, such as a hemodynamic response pattern (e.g. photoplethysmographic (PPG) signal), as will be described in more detail below. The output of the active optical modulating element 132a is therefore a time-varying modulated output light signal S2 that is coupled back into the phantom material 110 to simulate absorption/scattering variations within the phantom material 110 which is of great interest in diagnostics.

In a preferred implementation, the active optical modulating element 132a is a liquid crystal device (LCD) or other electrochromic device, configured to vary its opacity or transmittance at certain wavelengths according to an applied drive voltage Vd so as to mimic an absorption/scattering variation within the phantom material 110.

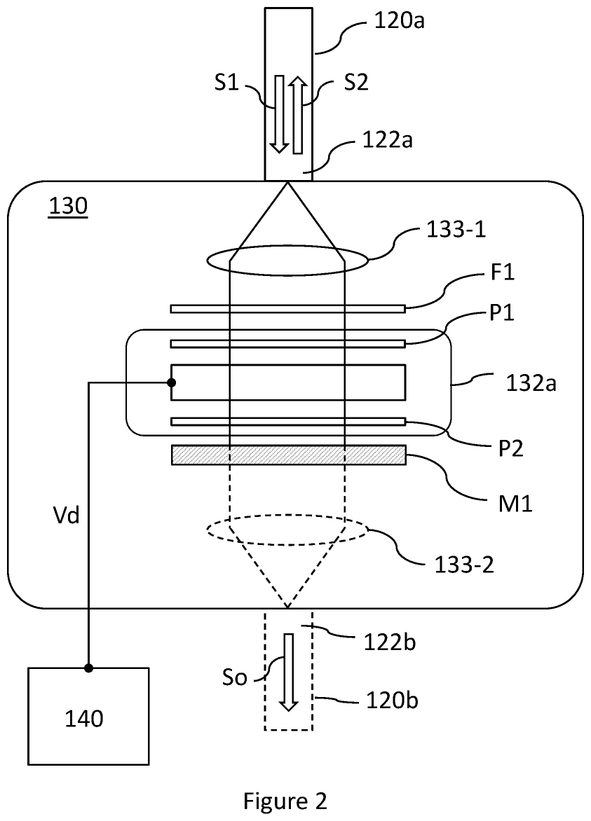

FIG. 2 shows an illustrative example of an optical modulation arrangement 130 comprising an LCD 132a. In this configuration, the optical modulation arrangement 130 comprises a collimating lens 133-1, an optical filter F1, an LCD 132a, and a mirror M1. The collimating lens 133-1 converts the diverging light beam from the second end 122a of a first light guide 120a into a collimated input beam S1 for illuminating the LCD 132a. The filter F1 is positioned in the illumination optical path between the lens 133-1 and the LCD for selecting the proper wavelength of light for measurement. The LCD 132a comprises a liquid crystal cell between two crossed polarisers P1, P2, and is arranged to receive the collimated input light beam S1 at normal incidence for which the LCD 132a is designed to operate. The transmittance of the LCD 132a is controllable by the drive voltage Vd provided by the driver 140 so as to control the intensity of the output light S2. The mirror M1 is positioned after the LCD 132a to reflect the collimated output light S2 back through the LCD 132a and onto the collimator 133-1 where it is coupled back into the second end 122a of the first light guide 120a. The collimated normal incidence input light eliminates the dependence of attenuation properties of the LCD 132a on the incident light angle and surround phantom material 110 properties, making the performance of the LCD 132a and the resulting modulation of light predictable and optimised.

With reference again to FIG. 2, in an alternative configuration, the output light S2 can be coupled into the second end 122b of a second light guide 120b (i.e. a different light guide), as illustrated by the dashed components. In this configuration, the mirror M1 is omitted and the optical arrangement 130 further comprises a second collimating lens 133-2 to couple the collimated output light S2 from the LCD 132a into the second end 122b of the second light guide 120b.

In an embodiment, the optical arrangement 130 comprises one or more light sources 134. At least one light source(s) 134 is used to produce locator light that can be selectively coupled into the second end 122 of the or each light guide 120 and emitted at the respective first end(s) 121 to indicate the location of the respective first end(s). In this way, when locator light is coupled to the second ends 122 of the light guides 120, the first ends 121 serve as optical beacons indicating their locations for alignment of the optical measurement instrument 10. The locator light is preferably visible light, but it can be NIR light depending on the instrument detector. Alternatively or additionally, the light source(s) 134 can be used for producing the output light used to simulate the desired optical property variation. In this case, the light source(s) may be configured to emit light matched to the source of the instrument or selected based on the particular optical effect being simulated. The light sources 134 may comprise a laser diode or light emitting diode, or other suitable electrically controllable light source. The light source(s) 134 are configured to produce the output light and/or the locator light in response to an input signal.

Tissue optical properties are wavelength dependent, and different biomarkers exhibit different absorption and scattering spectra. As a result, various diagnostic techniques use multiple wavelengths of incident light to obtain additional information that is used to extract the desired biological/physiological behaviours. For example, a well-known problem in NIR spectroscopy for biomedical applications is the coupling of the oxyhaemoglobin (HbO2) and deoxyhaemoglobin (Hb) absorption spectra for which it is now common practice, especially in pulse oximeters, to use two wavelengths to obtain separate information about each blood constituent (typically 660 or 760 nm is used for Hb and 830 nm or 940 nm is used for HbO2). Consequently, optical measurement instruments may be configured to provide simultaneous illumination at a plurality of wavelengths.

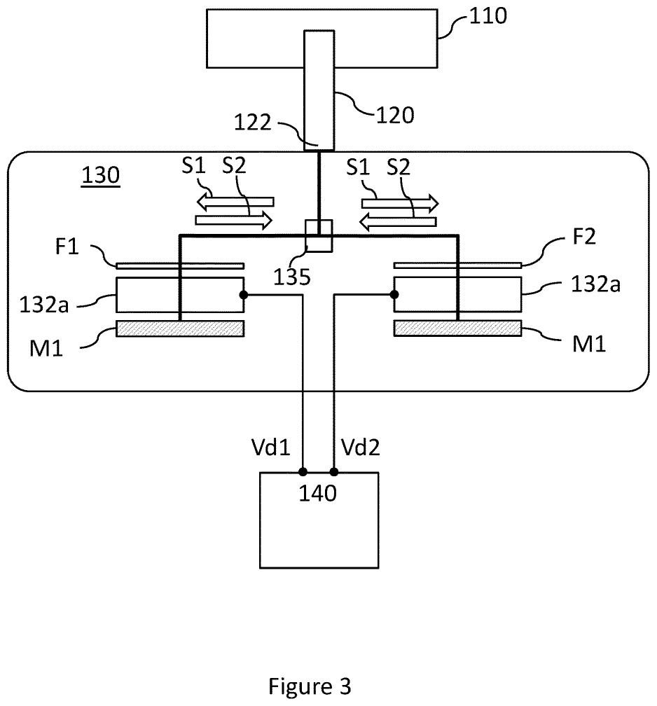

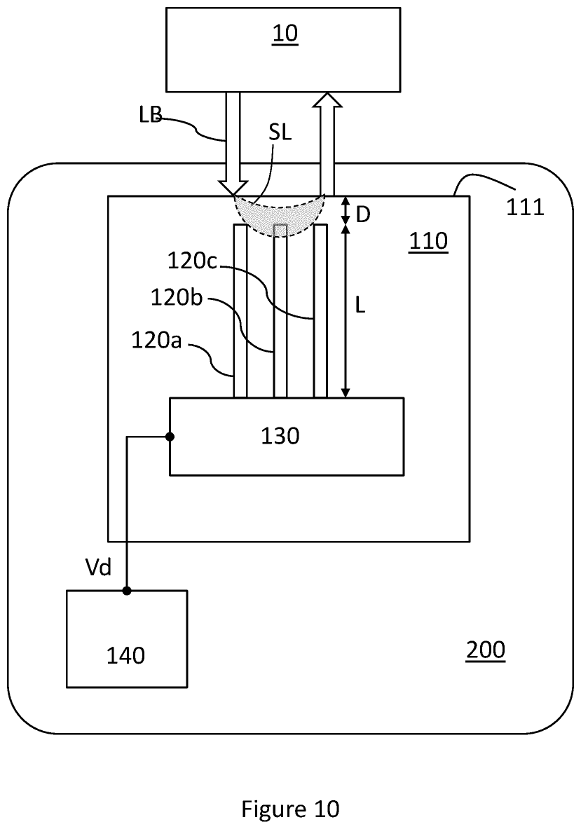

FIG. 3 shows an illustrative example optical modulation arrangement 130 comprising a plurality of LCDs 132a, each for a different selected wavelength of light. The configuration of each LCD 132a is the same as in FIG. 2 (some components are omitted for clarity), except that in this case each LCD 132a is paired with an optical filter F1, F2 for the respective selected wavelength. Each LCD 132a is driven by a respective drive voltage Vd1, Vd2 from the driver 140 which is dependent on the selected wavelength in order to mimic the desired absorption/scattering variation at that wavelength. The collimated input light beam S1 is split between the two LCDs 132a using a beam splitter or suitable dichroic mirror 135 (alternatively, different light guides 120 can be used for each different wavelength required, see e.g. FIG. 9). Although only two LCDs 132a are shown in FIG. 2, in general there can be as many LCDs 132a as there are wavelengths used in the measurement. Further, different light guides 120b can be used to send the output light S2 back to the phantom material 110 as illustrated in FIG. 2.

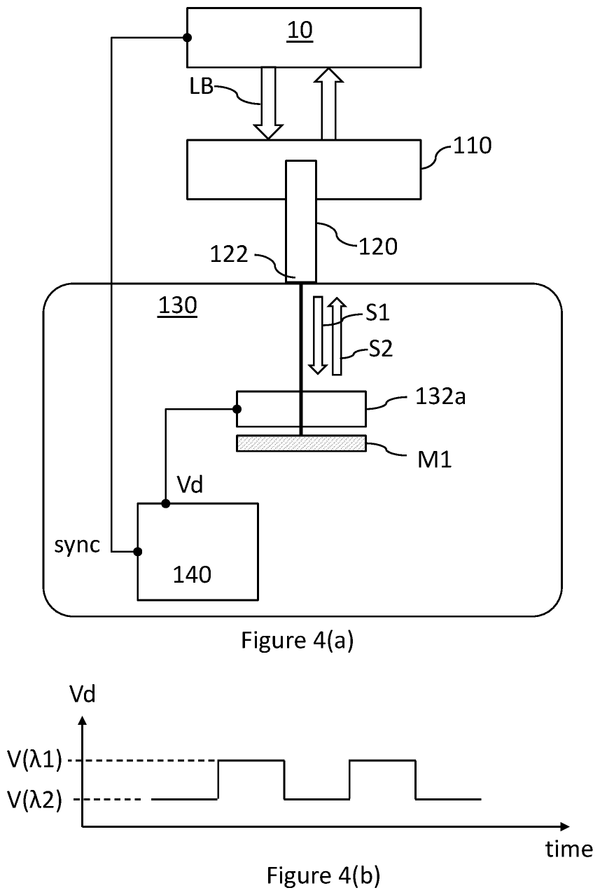

In the configuration of FIG. 3, the driver 140 or each LCD 132a is operated in a first “single colour” or “unsynced” mode whereby the drive voltage Vd and hence transmission of each LCD 132a is specific for the selected wavelength. A different drive signal Vd is provided to each LCD 132a based on the selected wavelength. FIG. 4(a) shows an alternative configuration for multiple wavelength illumination that uses only one LCD 132a. In this configuration, the optical filter F1 is omitted such that light of each different wavelength impinges on the LCD 132a. With reference to FIG. 4(b), the driver 140 or LCD 132a is operated in a second “multicolour” or “synced” mode whereby the drive voltage Vd and hence transmission of the LCD 132a switches periodically between different wavelength-dependent values Vd(λ1) and Vd(λ2) over time in a manner that is synchronised with the detection timing or data acquisition of the optical measurement instrument 10. In this way, when the optical measurement instrument 10 is detecting scattered light SL at the first wavelength λ1, the LCD 132a has a transmission/opacity required to simulate the desired absorption at λ1, and when the optical measurement instrument 10 is detecting scattered light SL at the second wavelength λ2, the LCD 132a has a transmission required to simulate the desired absorption at λ2. In this second configuration, the driver 140 synchronises the wavelength dependent drive voltage Vd to the optical measurement instrument 10 using a sync signal received from the optical measurement instrument 10, such as a TTL signal (see FIG. 4(a)).

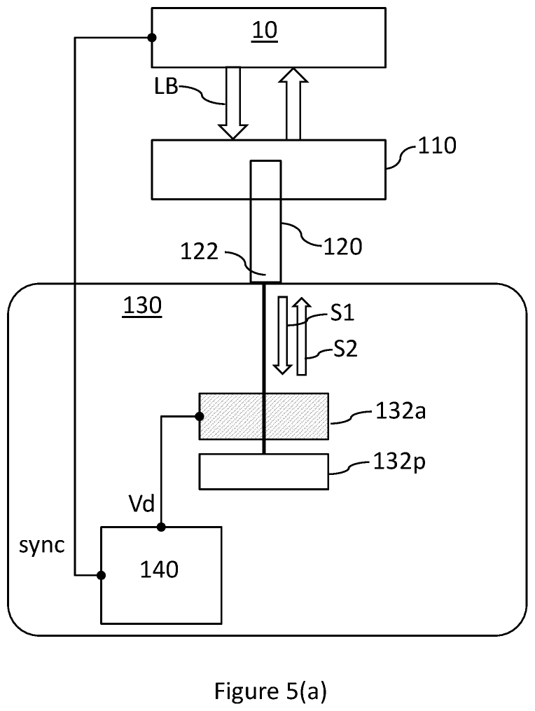

Although a specific arrangement is shown in FIGS. 2-4, due to the external modulation, the optical modulation arrangement 130 can be configured to produce the output light S2 in various ways depending on the specific optical modulating element 132 used. For example, the output light S2 may be transmitted, reflected, scattered, or emitted from the optical modulating element 132. An LCD 132a is not essential. By bringing light outside the phantom material 110, the light S1 can be actively modulated using other electro-optical modulators, electro-mechanical optical modulators such as a deformable mirror device (DMD), or simply by using a mechanical shutter device. With reference to FIG. 2, a configuration with a shutter device 132a in the place of an LCD 132a would look the same, and a configuration with a DMD 132a in the place of an LCD 132a would look the same save for omitting the mirror M1. A DMD or shutter device may be advantageous in certain applications, because more light can be coupled back into the phantom material 110. For example, an LCD can lose up to 50% of light due to the use of polarizers P1, P2 alone (see FIG. 5(b)).

Bringing the input light S1 outside of the phantom material 110 also allows for entirely new phantom configurations for biophotonic techniques. FIG. 5(a) shows an example configuration in which the one or more optical modulating elements 132 includes an active optical modulation element 132a and a “passive” optical modulating element 132p. The active optical modulating element 132a can be operate in synced or unsynced modes, as described above. The passive optical modulating element 132p comprises an optical medium configured to produce scattered or emitted light from the interaction of incident light beam SI with the optical medium and which can be collected and coupled back into the phantom material 110. In one example, the passive optical modulating element 132p comprises a Raman active material for mimicking Raman features within the phantom material 110. Upon illumination, the Raman active material produces output light S2 component with a Raman shifted wavelength (the Raman signal). In other examples, the passive optical modulating element 132p can comprise a fluorescent material or upconverting nanoparticles or a diffuse correlation solution. In this example, the electronically controllable modulating element 132a is positioned in front of the passive optical modulating element 132p to control the intensity of the incident light on it and thereby the strength of the output signal S2. A focusing lens can also be used to focus light onto or within the optical medium (see below).

FIG. 5(b) shows an example configuration with an active and passive optical modulating elements 132a, 132p in more detail. The optical arrangement 130 comprises a collimating lens 133-1, an optical filter F1, an LCD 132a, and focusing lens L1 and a passive optical modulating element 132p. Light coming out of the LCD 132a is focused onto or within the optical medium of the passive optical modulating element 132p, and output light S2 produced by the optical medium is collected by the same lens L1 in reflectance geometry. This produces a collimated output light beam S2 that is coupled back into the second end 122 of the light guide 120 by the collimating lens 133-1. It will be appreciated that for substantially transparent optical media, such as certain diffuse correlation solutions, the output light S2 can instead be collected in transmission geometry using an additional collection lens (not shown).

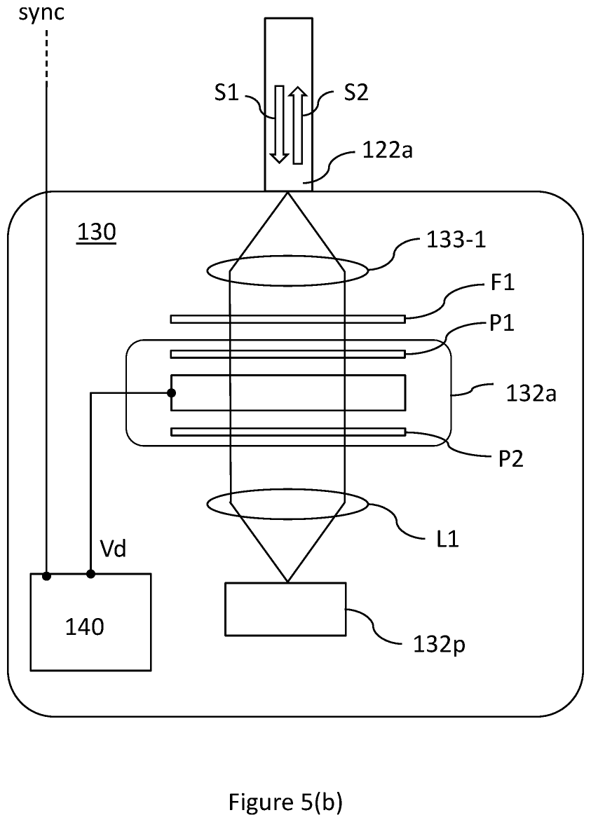

FIG. 6 shows another application of the external optical modulation system 130. In this configuration, the optical modulation system 130 comprises a passive optical modulating element 132p in the form of a sample cell 136 containing blood, and a mirror M1 for directing output light S2 from the sample cell back to the second end 122 of the light guide 120. This configuration can be used to make a preliminary measurement of phantom optical properties with real blood in the place of an active optical modulating element 132a such as an LCD, which can be used to calibrate the active optical modulating element 132a to produce absorption changes as seen in real blood. In one example, the sample cell 132p is connected to oxygen tank 136t for controlling the oxygen level in the blood.

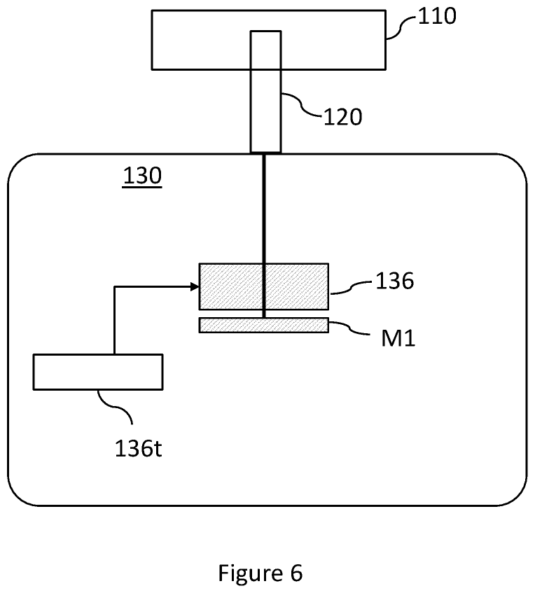

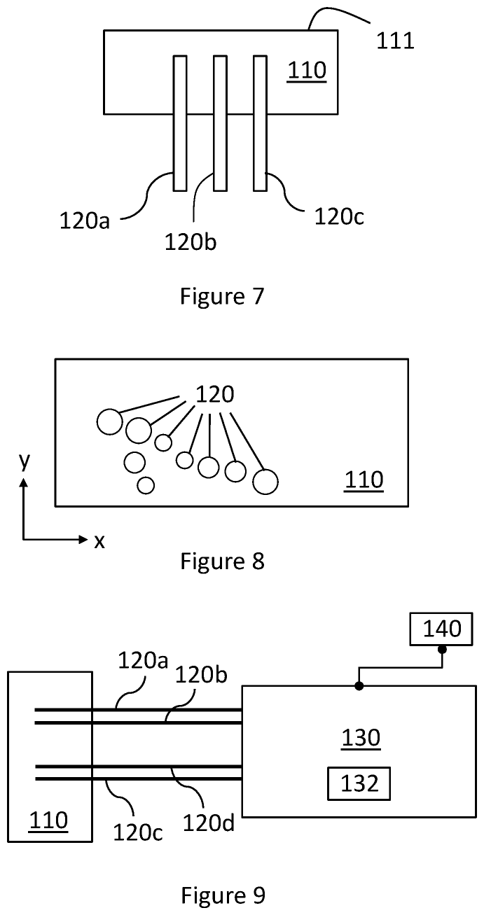

The first end 121 of the or each light guide 120 provides a localised spatial variation in the optical properties within the phantom material 110. The first ends 121 of the light guides 120 can therefore be arranged according to a known spatial response pattern. With reference to FIGS. 7 to 9, the system 100 can comprise a plurality of light guides 120. In a preferred implementation, the first ends 121 of each light guide 120 are located at different points in the phantom material 110 and/or spatially distributed according to a predefined spatial response pattern, such as an anatomical structure and the second ends 122 couple light onto a separate optical modulating element 132. With reference to FIG. 8, in one example, the first ends 121 of the light guides 120 can be arranged in a pattern to mimic the vessels plexus under the skin or other certain shapes present in biological tissue. In this configuration, for more realistic vessels network simulation, the light guides 120 can have different core diameters and/or the first ends 121 can be located at different depths beneath the surface 111.

With reference to FIG. 9, in some implementations where the optical modulation arrangement 130 comprises multiple optical modulating elements 132 for multi-wavelength illumination-detection techniques, the light guides 120 are arranged in one or more groups, wherein the first ends 121 of each light guide 120 in a group are substantially co-located at a point within the phantom material 110. In this configuration, each light guide 120 in a group is used to guide light to a different optical modulating element 132 for modulating light of a different wavelength. Preferably, the or each group of light guides 120 are configured as a fan-out bundle, the first ends 121 being part of the common end of the fan-out bundle.

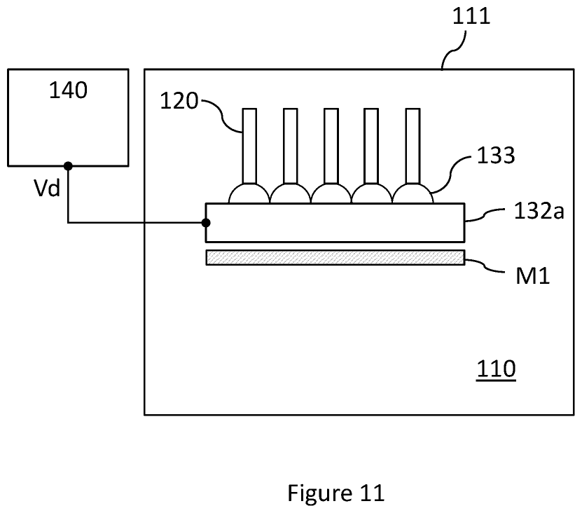

FIG. 10 shows a schematic diagram of a phantom system 200 according to a second embodiment to the invention. The system 200 comprises a phantom material 110, one or more light guides 120 and an optical arrangement 130 as described in the first embodiment, but in this second embodiment the optical arrangement 130 is embedded within the phantom material 110 to minimise the length L of the light guides 120 for (direct or indirect) time of flight (ToF) diffuse optical spectroscopy. ToF diffuse optical spectroscopy techniques measure ToF distributions of detected photons. The presence of light guides 120 increases the path length in the system 100, 200 and distorts the ToF. This can be corrected to a certain extent during ToF data analysis, but the shorter the light guides 120 the better as there will be less temporal distortion to the measured ToF curves.

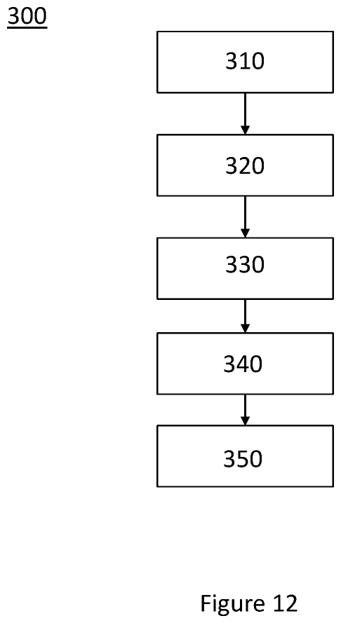

In preferred implementations, the light guides 120 have length of less than 5 cm, and the optical modulation arrangement 130 comprises an active optical modulating element 132a driven by a driver 140. FIG. 11 shows an example configuration of the system 200 comprising a plurality of light guides 120 coupled at their second ends 122 to a segmented LCD 132a via an array of lenses 133 to provide normal incident illumination. Each segment of the LCD 132a is individually controllable by a respective drive signal Vd from the driver 140, and effectively represents a separate optical modulating element. A mirror M1 is provided behind the LCD 132a to return output light S2 from the LCD 132a back to the respective light guide 120.

In each embodiment of the system 100, 200, the driver 140 is configured to apply the drive voltage Vd to the LCD 132a to produce the desired variation in absorption. The drive voltage Vd is pre-determined and is based on the wavelength of the light, at least one optical property of the target at that wavelength (i.e. the absorption coefficient μₐ) and a known temporal response pattern. The drive voltage values for a given wavelength can be calibrated and stored in, or provided to, the driver 140 to create the desired optical property (static or dynamic) of the phantom 110 for a given wavelength of light.

In implementations where the phantom system 100, 200 is configured to mimic hemodynamic response patterns, software/a computer program can be used to simulate an absorption variation in the target based in the following parameters related to the hemodynamics: the wavelength of incident light (in nm), the total saturation of oxygen in % (SpO₂), the total volume of blood (in μmol) (ΔtHb), the respiratory rate (min⁻¹), the heart rate(min⁻¹), and the perfusion index. The program can then calculate the values of Hb and HbO₂ (in μmol), the absorption coefficient μₐ (in cm¹) and the variation of absorption in time. Once the values of absorption at the incident wavelength are determined, a calibration factor is used to convert these values into voltages values needed to drive the LCD 132a by the driver 140. The result is a time-varying drive signal Vd. This is repeated for as many wavelengths as needed. The resulting drive signals are then stored in, or provided to, the driver 140. Where multiple LCDs 132a are operated in the single colour mode, two separate wavelength dependent drive signals Vd are determined. Where a single LCD 132a is operated in the multi-colour mode, the voltage values for each wavelength at each point in time are interleaved according to the sync signal from the optical measurement instrument 10 to produce a composite wavelength dependent time varying drive signal as shown in FIG. 4(b). The software/computer program can be on-board the driver 140, or run on a computing device connected to, or in communication with, the driver 140.

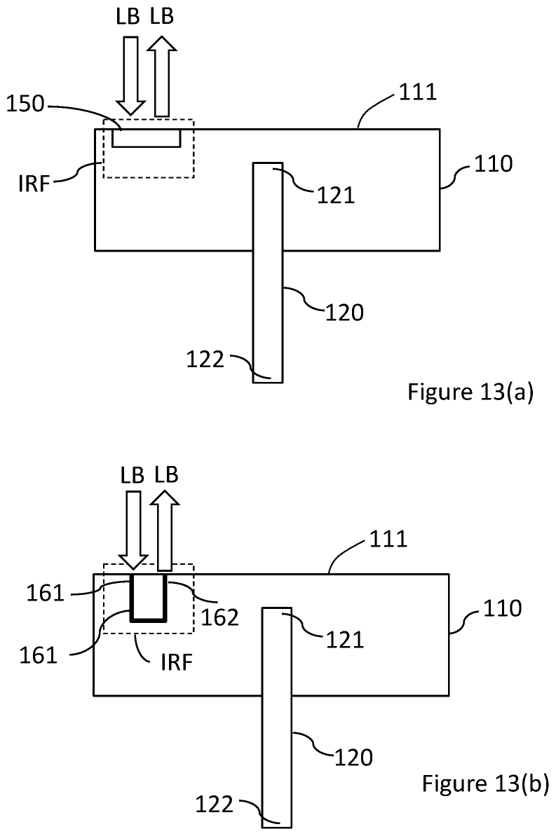

FIG. 12 shows a method 300 of calibrating an optical measurement instrument 10 configured to measure a target's response to illumination using the phantom system 100, 200 according to an embodiment of the invention. Step 310 comprises illuminating a surface 111 of a tissue phantom material 110 with a source beam. Step 320 comprises simulation a spatial and/or temporal modulation of an optical property within the tissue phantom material 110 according to a known spatial and/or temporal response pattern by coupling light into and/or out of the phantom material via one or more light guides 120 with a first end 121 embedded within the phantom material 110 beneath the surface 111. This comprises producing, at an optical arrangement 130 coupled to the second end of the or each light guide 120, output light; and sending the output light to the phantom material 110 via the one or more light guides 120. Step 330 comprises detecting, by the instrument 10, scattered light SL emitted from the tissue phantom material 110. Step 340 comprises determining a spatial and/or temporal response pattern based on the detected light. This may comprise using a software algorithm on-board or specific to the instrument 10. Step 350 comprises comparing the determined response pattern to the known response pattern to determine a calibration factor for the optical measurement instrument, e.g. to compensate for drift, distortion and/or other variations in the output/performance of optical measurement instrument. The method may further comprise calibrating the optical measurement instrument 10 using the determined calibration factor(s).

Step 320 preferably comprises receiving, at the optical arrangement 130, input light from the second end 122a of a first one 120a of the one or more light guides 120; modulating the input light to produce the output light; and coupling the output light into the second end 122a of the first one 120a or the second end 122b of a second one 120b of the one or more light guides 120 for sending to the phantom material 110. In some implementations, step 320 comprises reflecting the produced output light back to the second end 122a of the first one 120a of the one or more light guides 120.

In a preferred implementation, in step 320, modulating the input light comprises illuminating at normal incidence, with the input light, an active optical modulating element 132a having a variable optical property, and applying a time-varying drive signal Vd to the active optical modulating element 132a to selectively attenuate incident input light according to a known temporal response pattern. Preferably, the active optical modulating element 132a is or comprises a liquid crystal device.

Step 320 may further comprises determining a time-varying drive signal Vd based on a known temporal response pattern, a wavelength of incident light and at least one optical property of the target.

In step 350, the response pattern is preferably determined by the instrument's 10 built-in software algorithm(s). As such, step 350 compares the instrument's determined response values with calibrated values of the phantom system 100. The calibrated values of the phantom system 100 are determined by experimental testing in lab. This is achieved by benchmarking the phantom response in lab under controlled experimental settings (wavelength, power of source, detection system performance) with analytical or Monte Carlo model. These properties will remain the same over the phantom lifetime. In step 350, the tested optical measurement 10 instruments can use these calibrated phantom values to benchmark the tested instrument's 10 performance against absolute calibration values of lab settings and/or relative calibration values obtained by the tested instrument 10 under ideal conditions. In either case, this comparison can be used for calibration of instrument algorithm and hardware performance, correction of non-linearity seen in instrument performance, correction of biomarker quantification, for routine quality checks for maintenance and servicing, life-time surveillance of instrument, provide insights into stability, reproducibility, linearity of instrument.

The phantom system 100 and method 300 can be used for testing and calibrating a broad range of optical instruments, including but not limited to: (i) diffuse reflectance probe or imaging (superficial or limited depth imaging of tissue) systems: comparing detected scattering light signal from step 330 with known internal calibrated diffuse reflectance response of the phantom system 100 for given phantom properties (absorption, reduced scattering, wavelength, etc.); (ii) fluorescence system: comparing fluorescence light detected from the phantom material 110 to a known dynamic fluorescence response of the phantom system 100 and applying a calibration factor; (iii) Raman System: comparing Raman signal detected from phantom system 100 to known Raman response of phantom system 100 and applying calibration factor; (iv) diffuse correlation spectroscopy: compare the decorrelation (g2) values obtained from instrument determined response with internal calibrated values of the phantom system 100; and (v) GASMAS: compare the gas absorption coefficient evaluated by the instrument 10 with internal calibrated values of the phantom system 100.

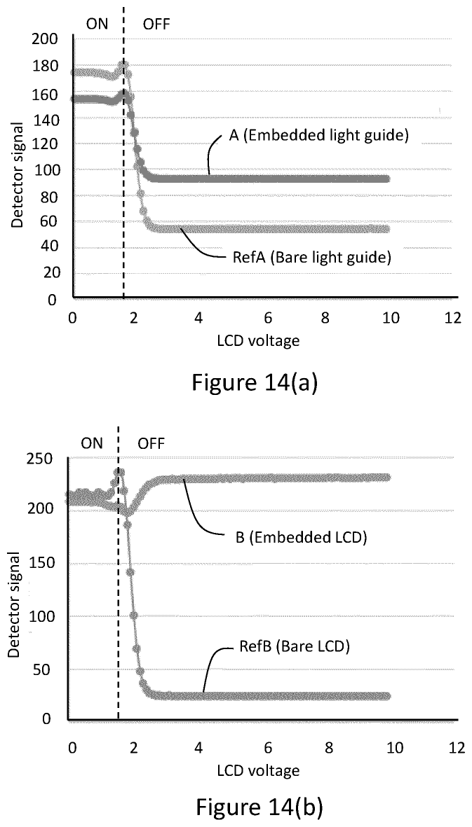

FIG. 13(a) and 13(b) show embodiments of the system 100 in which the phantom material 110 comprises an instrument response function (IRF) measurement zone. The IRF zone is configured to return incident light LB to the instrument 10 for detection without having interacted with the phantom material 110 for detection by the instrument 10. In FIG. 13(a), the IRF zone comprises a reflective element 150 provided at/on the input surface 111 of the phantom material 110 for reflecting incident source light LB back to the instrument 10. The reflective element 150 can be an object such as a mirror or the like which is embedded in the input surface 111, or it can be a reflective coating or layer provided on the input surface 111. FIG. 13(b) shows an alternative embodiment in which the IRF zone comprises an embedded IRF light guide 160 to return the incident source light LB. The IRF light guide 160 comprises a first end 161 positioned at a first location at the input surface 111 for collecting incident source light LB and a second end 162 positioned at a second location on the input surface 161 for emitting source light transmitted from the first end 161. The first and second ends 161, 162 are preferably oriented substantially normal to the input surface 111.

### Experimental Data

To demonstrate the benefit of the embedded light guide approach of the present invention to the embedded LCD approach of the prior art, the following experiment was performed. Equivalent phantom systems based on the embedded light guide approach (phantom A) and embedded LCD approach (phantom B) were prepared. Phantom A corresponded to the embodiment of FIG. 2, comprising a light guide 120 with a first end 121 embedded at a depth of D=5 mm beneath the input surface 111 of a phantom material 110 and a second end 122 located outside of the phantom material 110. Input light provided from the second end 122 is coupled to an LCD 132a at normal incidence via a collimating lens 133 and a spectral filter F1, and output light transmitted through the LCD 132 is reflected, via a mirror M1, back through the LCD 132a and into the second end 122 for transmitting to the phantom material 110. In phantom B, the same LCD 132a was embedded at a depth of D=5 mm beneath the input surface of a phantom material. In both cases, the phantom material had an absorption coefficient 0.1 cm⁻¹ and reduced scattering coefficient 10 cm⁻¹. A light source was used to illuminate the input surface of the phantom material with source light at a wavelength of 660 nm (which is typically used in pulse oximeters and other biophontonic devices) and diffused/scattered light from the phantom was collect by a camera detector while the drive voltage Vd applied to the LCD 132a was varied. Reference measurements were also made on the same systems without the phantom material to demonstrate the ideal LCD response (reference systems RefA and RefB).

FIG. 14(a) and 14(b) shows the detector signal as a function of drive voltage Vd applied to the LCD 132a for phantom systems A and B and reference systems RefA and RefB, respectively. In the absence of a phantom material 110 both light guide reference system RefA and the LCD reference system RefB exhibit substantially the same behaviour, whereby increasing drive voltage from the ON state (low drive voltage) to the OFF state (high drive voltage) decreases the light reaching the detector. When embedded in phantom material 110, the light guide phantom system A behaves as expected, showing a reduction in detector signal (photon count) from the ON state (low drive voltage) but with a reduction in contrast between the detector signals for the ON and OFF states due to diffuse reflectance of photons from phantom material 110. However, in the embedded LCD phantom system B, the presence of phantom material leads to inverse behaviour of LCD, i.e. increasing drive voltage increases the detector signal, and dramatically reduced contrast between the detector signals for the ON and OFF states. This can be related to change in direction of photons impinging on LCD 132a due to diffusion of photons in phantom material 110, as LCD performance is a function of angle of incident of light. This example shows why current state-of-the-art LCD-based dynamic phantoms are sub-optimal for biophotonics phantom purposes. In contrast, because the angle of light emitted from the second end 122 of the light guide 120 and impinging on the optical modulating elements 132 of the optical arrangement 130 is always constant, and can be collimated by optics before interacting with modulator 132, the performance, predictability and reliability of the optical modulating elements 132, and the resulting simulated optical property variation within the phantom material 110 is dramatically improved.

The project leading to this patent application has received funding from the European Union's Horizon 2020 research and innovation programme under grant agreement No 101017113.

From reading the present disclosure, other variations and modifications will be apparent to the skilled person. Such variations and modifications may involve equivalent and other features which are already known in the art, and which may be used instead of, or in addition to, features already described herein.

Although the appended claims are directed to particular combinations of features, it should be understood that the scope of the disclosure of the present invention also includes any novel feature or any novel combination of features disclosed herein either explicitly or implicitly or any generalisation thereof, whether or not it relates to the same invention as presently claimed in any claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub-combination.

For the sake of completeness it is also stated that the term “comprising” does not exclude other elements or steps, the term “a” or “an” does not exclude a plurality, and any reference signs in the claims shall not be construed as limiting the scope of the claims.

1. A tissue phantom system, comprising:
a tissue phantom material having known optical properties and an input surface for receiving incident source light from an optical measurement instrument;
one or more light guides having a first and second end, wherein the first end of the or each light guide is embedded within the phantom material at a respective location beneath the input surface to transmit light to and/or from the respective location for simulating an optical property variation within the tissue phantom material; and
an optical arrangement coupleable to the second end of the or each light guide, the optical arrangement configured to:
receive input light from the second end of a first one of the one or more light guides;
modulate the input light to produce output light; and
couple the output light into the second end of the first one or a second one of the one or more light guides for transmitting to the phantom material.

2. (canceled)

3. The system of claim 1, wherein the optical arrangement and the second end of the or each light guide are located outside of the phantom material.

4. The system of claim 1, wherein the optical arrangement and the second end of the or each light guide is also embedded within the phantom material; and, optionally preferably, wherein the length of the or each light guide is less than 5 cm.

5 to 39. (canceled)

40. The system of claim 1, wherein the optical arrangement is configured to simulate at least one of the following optical properties: absorption, scattering, emission, fluorescence, bioluminescence, Raman scattering, and dynamic lighting scattering; and/or wherein the simulated optical property is static or time-varying.

41. The system of claim 1, wherein the optical arrangement is configured to modulate the output light according to a predefined temporal response pattern to simulate a temporal variation in an optical property of the phantom material.

42. The system of claim 1, wherein the optical arrangement comprises one or more of the following:
(i) one or more light sources for producing locator light, and wherein the optical arrangement is configured to selectively couple the locator light into the second end of the or each light guide to indicate the location of the first end of the or each light guide;
(ii) one or more light sources for producing output light;
(iii) a reflective element for directing the output light back to the second end of the first one or the second one of the one or more light guides; and
(iv) one or more optical modulating elements having a fixed or variable optical property, the one or more optical modulating elements configured to produce the output light by the interaction of incident input light with the respective optical modulating element.

43. The system of claim 1, wherein the optical arrangement comprises one or more optical modulating elements, the one or more optical modulating elements comprising an active optical modulating element having an electrically controllable optical property, the active optical modulating element configured to selectively attenuate incident input light according to an input drive signal to thereby provide the output light.

44. The system of claim 43, wherein the active optical modulating element comprises: an electrochromic material configured to vary the transmission of incident input light according to an input drive signal; or a liquid crystal device.

45. The system of claim 1, wherein:
the one or more light guides is a plurality of light guides;
the active optical modulating element comprises a segmented liquid crystal device, wherein the second end of each light guide is arranged to provide input light to a different segment or group or segments of the liquid crystal device, and wherein the segmented liquid crystal device is configured to selectively attenuate incident input light received from each light guide in response to an input drive signal applied to each respective segment or group of segments;
the optical arrangement comprises a collimating lens array for coupling input light out of the second end of each respective light guide, and wherein the segmented liquid crystal device is arranged to receive collimated input light from each light guide at normal incidence; and optionally or preferably, wherein
the optical arrangement comprises a reflective element arranged to direct output light transmitted through the segmented liquid crystal device back through the segmented liquid crystal device and the collimating lens array for coupling back into the second ends of the light guides.

46. The system of claim 43, wherein the active optical modulating element is or comprises an electro-mechanical modulator configured to vary the reflectance of incident input light according to an input drive signal; and, optionally or preferably, wherein the active optical modulating element is or comprises a deformable mirror device; or
wherein the active optical modulating element is or comprises a mechanical shutter device configured to vary the transmission of incident input light according to an input drive signal.

47. The system of claim 43, wherein the active optical modulating element is arranged to receive the input light at normal incidence.

48. The system of claim 1, wherein the optical arrangement comprises one or more collimators for coupling input light out of and/or output light into the second end of the or each respective light guide; or
wherein the second end of the or each light guide comprises a collimator for coupling input light out of and/or output light into the light guide.

49. The system of claim 1, wherein the optical arrangement comprises one or more optical modulating elements, the one or more optical modulating elements comprising a passive optical modulating element, the passive optical modulating element including an optical medium configured to produce scattered and/or emitted output light from the interaction of incident light with the optical medium.

50. The system of claim 1, wherein the input light comprises a plurality of wavelengths, and wherein the optical arrangement comprises:
a plurality of optical modulating elements for modulating the input light at each respective wavelength; and
a plurality of spectral filters to select the wavelength of input light incident upon each respective optical modulating element.

51. The system of claim 43, comprising a driver configured to apply an input drive signal to the active optical modulating element to produce the output light according to a predefined static or temporal response pattern, wherein the input light incident upon the active optical modulating element comprises a plurality of wavelengths, and the driver is configured to apply an input drive signal to the active optical modulating element that is a wavelength-dependent time varying drive signal based on a synchronising signal received from the optical measurement instrument, such that the modulation of the incident input light provided by the active optical modulating element is synchronised with a detection timing of the optical measurement instrument.

52. The system of claim 1, comprising a plurality of said light guides, and wherein:
(i) the first ends of each light guide are located at different positions in the phantom material;
(ii) the first ends of each light guide are located at a range of different depths beneath the input surface of the phantom material;
(iii) the first ends of each light guide are spatially distributed according to a predefined spatial response pattern, preferably a vessels plexus pattern; and/or
(iv) the plurality of light guides have a range of different core diameters;
(v) the plurality of light guides are arranged in one or more groups, wherein the first ends of each light guide in a given group are substantially co-located at a point within the phantom material, and preferably, wherein each given group of light guides are configured as a fan-out bundle, the first ends of the group being part of a common end of the fan-out bundle.

53. The system of claim 1, wherein the phantom material comprises an instrument response function (IRF) measurement zone, wherein the IRF measurement zone comprises:
a reflective element provided at or on the input surface of the phantom material for reflecting incident source light; or
an embedded light guide having a first end positioned at a first location at the input surface of the phantom material for collecting incident source light and a second end positioned at a second location at the input surface for emitting source light transmitted from the first location.

54. A method of calibrating or testing an optical measurement instrument configured to measure a target's response to illumination, comprising:
illuminating an input surface of a tissue phantom material with source light, the phantom material having a first end of one or more light guides embedded at a respective location beneath the input surface;
simulating an optical property variation within the tissue phantom material according to a known response pattern by transmitting light to and/or from the respective location beneath the input surface via the one or more light guides; and
detecting scattered light emitted from the tissue phantom material,
wherein simulating the optical property variation comprises:
receiving, at an optical arrangement coupled to a second end of the or each light guide, input light from the second end of a first one of the one or more light guides;
modulating the input light to produce the output light; and
coupling the output light into the second end of the first one or a second one of the one or more light guides for transmitting to the phantom material.

55. The method of claim 54, further comprising:
determining a spatial and/or temporal response pattern based on the detected light;
comparing the determined response pattern to the known response pattern; and
determining one or more calibration factors for the optical measurement instrument based on the comparison of the determined response pattern and the known response pattern.

56. The method of claim 54, comprising:
measuring an instrument response function (IRF) by:
illuminating a reflective element provided at or on the input surface of the phantom material with source light, and detecting the source light reflected by the reflective element; or
illuminating the first end of a IRF light guide provided at a first location on the input surface of the phantom material with source light, and detecting the source light emitted from a second end of the IRF light guide provided at a second location on the input surface of the phantom material; and
determining one or more calibration factors for the optical measurement instrument based on the measured IRF.
